(19) United States
(12) Patent Application Publication
(10) Pub. No.: US 2022/0370759 A1
(43) Pub. Date: Nov. 24, 2022

(54) Catheter

(71) Applicant: RAUMEDIC AG, Münchberg (DE)

(72) Inventor: Björn GLÄSEL, Kirchenlamitz (DE)

(73) Assignee: RAUMEDIC AG, Münchberg (DE)

(21) Appl. No.: 17/770,540

(22) PCT Filed: Oct. 8, 2020
(86) PCT No.: PCT/EP2020/078221

§ 371 Date: Apr. 20, 2022

(30) Foreign Application Priority Data
Oct. 22, 2019 (DE) 10 2019 216 253.3
Oct. 22, 2019 (DE) 10 2019 216 254.1

(51) Int. Cl. A61M 25/00; B26D 7/08; B29C 57/10

(52) U.S. Cl. CPC A61M 25/0015; A61M 25/0068; A61M 25/008; B26D 7/086; B29C 57/10; A61M 2025/0046; A61M 2025/0081

(57) ABSTRACT

A catheter has a catheter tube made of plastic with a closed catheter tip. At least one passage opening is formed in a section of a sheath wall adjacent to the catheter tip. The passage opening merges into the outer sheath wall of the catheter tube via rounded edge regions. The result is a catheter whose insertion into a body cavity is facilitated.

### CROSS-REFERENCES TO RELATED APPLICATIONS

This application claims the priorities of German Patent Applications, Serial No. DE 10 2019 216 253.3, filed Oct. 22, 2019 and Serial No. DE 10 2019 216 254.1, filed Oct. 22, 2019, the contents of which are incorporated herein by reference in their entirety as if fully set forth herein.

### FIELD OF THE INVENTION

The invention relates to a catheter.

### BACKGROUND OF THE INVENTION

Such a catheter is known from DE 691 00 816 T2. DE 20 2008 017 390 U1 discloses a catheter and an apparatus for manufacturing such a catheter. DE 10 2011 104 840 A1 discloses a method and an apparatus for ultrasonic punching. US 2016/0184551 A1 discloses a urinary catheter having a soft tip. DE 69 504 104 C2 discloses a method for manufacturing a soft tip. DE 35 06 738 A1 discloses a method for forming a soft, deformable tip at the distal end of a tubular thermoplastic or elastomeric catheter. DE-PS 660 774 discloses a method for manufacturing urinary catheters.

### SUMMARY OF THE INVENTION

It is an object of the present invention to further develop a catheter of the type mentioned above in such a way that its insertion into a body cavity, in particular into a ureter, a blood vessel or a ventricle, is facilitated.

This object is achieved according to the invention by a catheter having a catheter tube made of plastic with a closed catheter tip, having at least one passage opening in a section of a sheath wall adjacent the catheter tip, wherein the passage opening merges into an outer sheath wall of the catheter tube via rounded edge regions, wherein the passage opening is manufactured by ultrasonic punching.

According to the invention, it has been found that in the region of a catheter tip it is not absolutely necessary to route a line path axially to the catheter tip, but that it is possible to design the catheter tip in a closed manner and to provide a line path via a passage opening in a sheath wall section adjacent to the catheter tip. The catheter tip can then be designed in particular without an edge region surrounding an opening, which enables designs of the catheter tip to facilitate the insertion of the catheter. The catheter may be used, for example, as a urinary catheter or as a ventricular catheter. The rounded edge regions of the at least one passage opening result in the fact that the sheath wall-side passage opening in turn does not have any disturbing edge regions that could cause problems during insertion. In particular, an atraumatic catheter can be realized. The catheter can have a plurality of passage openings, for example two passage openings, three passage openings, four passage openings, five passage openings or an even greater number of passage openings. The passage opening in the section of the sheath wall adjacent to the catheter tip is manufactured by ultrasonic punching. Manufacture by ultrasonic punching has been proven in practice. Such a manufacturing process for the at least one passage opening with outer rounded edge regions is particularly suitable for mass production.

According to the invention, the above-mentioned object is further achieved by a catheter having a catheter tube made of plastic with a closed catheter tip, having a passage opening in a sheath wall section adjacent to the catheter tip, wherein the catheter tip is formed on an end section of the catheter tube adjacent to the catheter tip, wherein the catheter tip is made of a plastic material the Shore hardness of which is lower than that of the adjacent end section, wherein in the region of the end section, a diameter of the catheter tube is reduced compared to the other catheter tube having an inner lumen, wherein the inner lumen of the catheter tube is configured to be closed in the region of the end section (8).

According to the invention, it has been found that a soft catheter tip is advantageous for facilitating an insertion of the catheter. Not only the catheter tip itself, but also the end section of the catheter tube can be of closed design. The reduced diameter of the catheter tube in the region of the end section compared to the other diameter of the catheter tube having the inner lumen facilitates an insertion of the catheter, since a volume displaced thereby is reduced. In the region of the end section, the inner lumen of the catheter is configured in a closed manner so that there is no uncontrolled exchange of media between the inner lumen and the catheter environment.

Materials such as thermoplastic polyurethane elastomer (TPU) or polyurethane (PUR) or PVC have proven particularly useful for medical catheters.

A slidable coating of the catheter tube further facilitates the insertion of the catheter.

Shore hardnesses of the catheter tip in the range between 40 Shore A and 70 Shore A have proven to be effective. The Shore hardness of the catheter tip may be in the range of 50 Shore A, of 55 Shore A, or of 60 Shore A. The catheter tip may be made of PVC or a thermoplastic elastomer.

Shore hardnesses in the range between 60 Shore A and 90 Shore A for the adjacent end section of the catheter tube have also proven to be effective. The Shore hardness of the adjacent end section may be in the range of 65 Shore A, of 70 Shore A, of 75 Shore A, of 80 Shore A, or of 85 Shore A. The adjacent end section may be made of harder PVC compared to the catheter tip or made of a TPE.

An end section which is made of drawn material of the catheter tube can be manufactured inexpensively and in a manner suitable for mass production.

An end section which is pressed to increase its circumference may be pre-formed such that an insertion of the catheter tip is facilitated due to the shape. In addition, the enlargement of the circumference of the end section results in a form fit of the end section with the catheter tip molded thereon.

A passage opening in the end section allows a form fit with the formed catheter tip.

A catheter tip which is formed as an overmolding component around the end section can be manufactured in a manner suitable for mass production.

The catheter tip is manufactured by overmolding the end section. Manufacturing specifics known from 2K technology can be used here.

The advantages of the manufacturing method of a catheter
having a catheter tube (2) made of plastic with a closed catheter tip (7; 12),
having a passage opening (4) in a sheath wall section adjacent to the catheter tip (7; 12),
wherein the catheter tip (7; 12) is formed on an end section (8; 10; 14) of the catheter tube (2) adjacent to the catheter tip (7; 12),
wherein the catheter tip (7; 12) is made of a plastic material the Shore hardness of which is lower than that of the adjacent end section (8; 10; 14),
wherein in the region of the end section (8; 10; 14), a diameter of the catheter tube (2) is reduced compared to the other catheter tube (2) having an inner lumen (4a),
wherein the inner lumen (4a) of the catheter tube (2) is configured to be closed in the region of the end section (8), correspond to those already explained above in connection with the catheter. Such catheter may also have one of the other embodiments discussed above.

Examples of embodiments of the invention are explained in more detail below with reference to the drawing.

### BRIEF DESCRIPTION OF THE DRAWING

FIG. 1 shows a perspective view of a section of a catheter tube of a catheter having a passage opening in a sheath wall section adjacent to a catheter tip;

FIG. 2 shows an axial section through the catheter tube section according to FIG. 1 in the region of the sheath wall-side passage opening;

FIG. 3 shows an axial section through an end section of the catheter tube having a closed catheter tip which is molded on an end section of the catheter tube adjacent to the passage opening and is made of a plastic material with lower Shore hardness compared to the end section;

FIGS. 4 and 5 each show in an illustration similar to FIG. 3, further embodiments of the catheter tube with a molded-on catheter tip made of plastic material of lower Shore hardness;

FIG. 6, shows a flow chart of a method for manufacturing a catheter tube having a catheter tip according to FIG. 5; and

FIGS. 7 and 8, in illustrations similar to FIGS. 3 to 5, show further embodiments of a catheter tip molded on a closed end section of a catheter tube.

### DESCRIPTION OF THE PREFERRED EMBODIMENTS

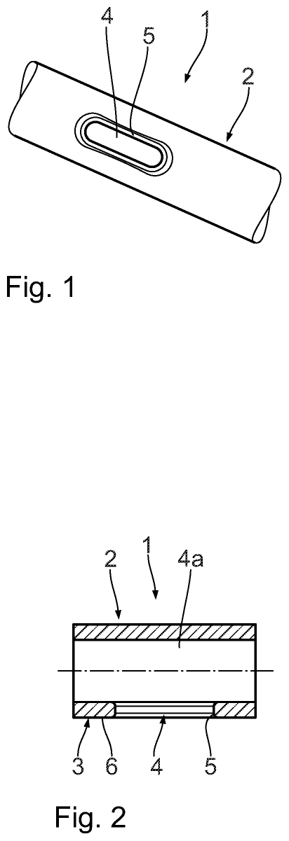

A catheter 1, of which only one portion of a catheter tube 2 is shown in FIG. 1, serves as a medical catheter, for example, as a urinary catheter for establishing a fluid connection with the interior of a patient's urinary bladder, or as a ventricular catheter for ventricular pressure equalization.

The catheter tube 2 is made of plastic material. Material variants for the catheter tube 2 are a thermoplastic polyurethane elastomer (TPU), a polyurethane (PUR), PVC or another thermoplastic elastomer (TPE). Silicone may also be used as a material for the catheter tube 2.

A Shore hardness of the catheter tube 2 is in the range between 60 and 90 Shore A. This Shore hardness of the catheter tube 2 may, for example, be in the range of 65 Shore A, of 70 Shore A, of 75 Shore A, of 80 Shore A or of 85 Shore A. The catheter tube 2 may be provided with a slidable coating. Vaseline may be used as a slidable coating. In order to create a slidable coating, a silicatization step can be performed in case silicone is used as the material for the catheter tube 2.

The catheter tube 2 may have an antimicrobial coating as an alternative to or in addition to the slidable coating. A coating of the catheter tube 2 may simultaneously serve as both a slidable coating and an antimicrobial coating.

In a section of a sheath wall 3 (cf. FIG. 2) adjacent to a catheter tip of the catheter 1 not shown in FIGS. 1 and 2, the catheter 1 has at least one passage opening 4. Fluid can pass from an inner lumen of the catheter tube 2 to the outside via the passage opening 4 and be discharged via a surrounding region of the catheter tube 2. The passage opening 4 merges into an outer sheath wall 6 of the catheter tube 2 via a rounded edge region 5.

The passage opening 4 and in particular the rounded edge region 5 are produced by ultrasonic punching.

The rounded edge portion 5 is present around an entire circumference of the passage opening 4.

Due to the rounded edge region 5, the catheter tube 2 can be inserted atraumatically in the region of the passage opening 4, for example into a ureter of the patient or also into a ventricle.

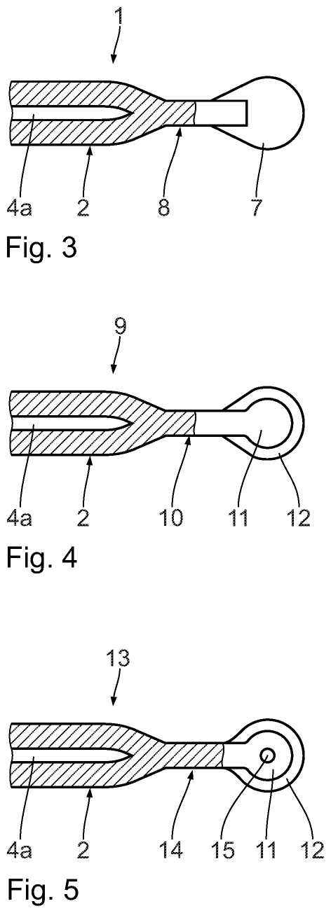

FIG. 3 shows an embodiment of a catheter tip 7 of the catheter 1 which is molded on an adjacent, that is tip-side end section 8 of the catheter tube 2. The end section 8 of the catheter tube 2 is made of drawn material of the catheter tube. In the region of the end section 8, the diameter of the catheter tube 2 is reduced compared to the other catheter tube 2 having the inner lumen 4a. In the region of the end section 8, the inner lumen 4a of the catheter tube 2 is configured in a closed manner. The catheter tube 2 is also closed in the region of the catheter tip 7.

The catheter tip 7 is made of a plastic material whose Shore hardness is lower than that of the adjacent end section 8 of the catheter tube 2.

The Shore hardness of the catheter tip 7 is in the range between 40 Shore A and 70 Shore A. This Shore hardness may be in the range of 50 Shore A, 55 Shore A or 60 Shore A. The catheter tip 7 can be manufactured of PVC or a thermoplastic elastomer. In principle, all soft thermoplastics are suitable as a material base for the catheter tip 7.

An outer diameter of the catheter 1 is larger in the region of the catheter tip than in the region of the adjacent end section 8 of the catheter tube 2.

The catheter tip 7 is formed as an overmolding component around the end section 8 of the catheter tube 2.

In the manufacture of the catheter 1, the catheter tube 2 is first extruded. Then the end section 8 is closed, which is carried out by drawing out the extruded material of the catheter tube 2 and, if necessary, twisting the drawn-out material. Subsequently, the catheter tip 7 is molded onto the end section 8 by overmolding.

Due to the softer catheter tip 7 and the spherical shape of the catheter tip 7, an atraumatic insertion of the catheter 1, for example, into a ureter or also into a ventricle is possible.

With reference to FIG. 4, a further embodiment of a catheter 9 which can be used instead of the catheter 1 is explained below. Components and functions corresponding to those already explained above with reference to FIGS. 1 to 3 bear the same reference numerals and are not discussed again in detail.

In the catheter 9, an end section 10 of the catheter tube 2 is pressed to enlarge a tip-side circumference of the end section 10. A tip-side end region 11 of the end section 10 having a spherical cross-section is obtained. This end region 11 having an enlarged cross section is manufactured by hot pressing the end section 10 before molding on a catheter tip 12. The catheter tip 12 of the catheter 9 is molded to the end region 11 of the end section 10 by overmolding, resulting in a layer on the end region 11 of a plastic material with lower Shore hardness, corresponding to what has been explained above regarding FIG. 3. The spherically pressed end region 11 results in a positive fit with the molded-on catheter tip 12, so that the latter does not undesirably become detached in the end region 11.

With reference to FIG. 5, a further embodiment of a catheter 13 which can be used instead of the catheter 1 is explained below. Components and functions corresponding to those already explained above with reference to FIGS. 1 to 4 bear the same reference numerals and are not discussed again in detail.

In the catheter 13, an end section 14 of the catheter tube 2 has a punched-in passage opening 15 in the end region 11. When the material of the catheter tip 12 is overmolded around the end region 11 of the end section 14, an additional form fit results in the catheter 13 due to the fact that the softer plastic material of the catheter tip 12 penetrates through the passage opening 15. This results in a captive catheter tip 12 in the catheter 13.

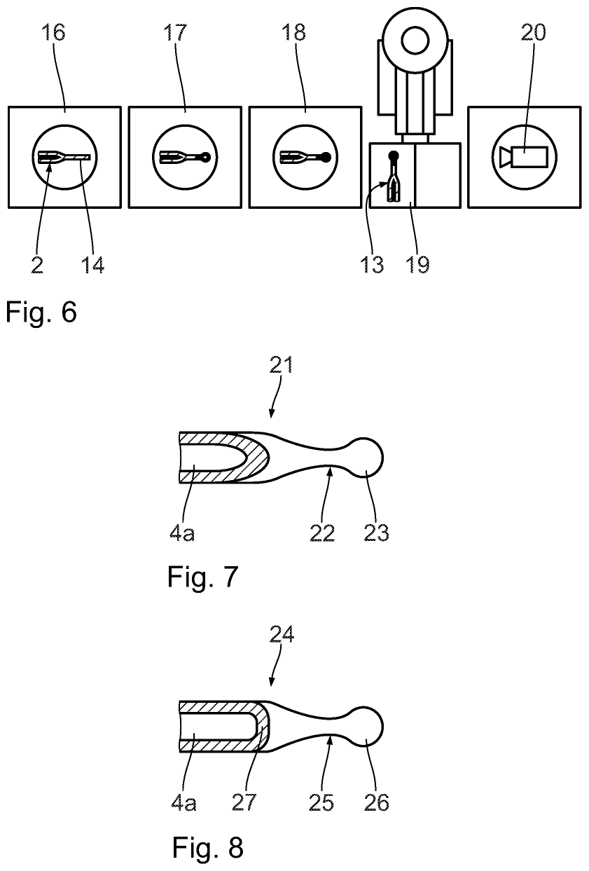

FIG. 6 shows a flow chart in the manufacture of the catheter 13.

In a drawing step 16, the end section 14 of the catheter tube 2 is drawn, twisted and closed.

In a hot pressing step 17, the end region 11 of the end section 14 is formed.

In a punching step 18, the passage opening 15 is inserted into the end region 11.

In an overmolding step 19, the catheter tip 12 is created by overmolding the end region 11. This is done by single cavity molding.

In addition, at least one more passage opening 4 is provided for in the catheter 13 by means of ultrasonic punching, as explained above.

The overmolding step 19 in particular, but also the other method steps in the manufacture of the catheter 13, are monitored with the aid of a camera 20.

With reference to FIGS. 7 and 8, two further embodiments of a catheter are described below which can be used instead of the catheters described above. Components and functions corresponding to those already explained above with reference to FIGS. 1 to 6 bear the same reference numerals and are not explained again in detail.

The catheter 21 according to FIG. 7 has an end section 22 with a pressed end region 23 for generating a spherical catheter tip comparable to the end region 11 of the catheter 9 according to FIG. 4.

Instead of an end region having a spherical cross-section, the catheter 24 according to FIG. 8 has an end section 25 having an end portion 26 with a club-shaped cross-section. In addition, a closure region of the lumen 4a of the catheter 24 does not taper to a point as in the other catheters described above, but is round (compare the closure region 27 in the FIG. 8). When the end section 25 is closed, the extruded material of the catheter tube 2 of the catheter 24 is less drawn than twisted.

In the case of catheters 21 and 24, the respective end section 22, 25 may itself constitute the catheter tip. In addition, a catheter tip made of a softer plastic material can also be molded on here, as already explained above in connection with the embodiments according to FIGS. 1 to 6.

1. A catheter (1; 9; 13; 21; 24)
having a catheter tube (2) made of plastic with a closed catheter tip (7; 12; 23; 26),
having at least one passage opening (4) in a section of a sheath wall (6) adjacent the catheter tip (7; 12; 23; 26),
wherein the passage opening (4) merges into an outer sheath wall (6) of the catheter tube (2) via rounded edge regions (5),
wherein the passage opening (4) is manufactured by ultrasonic punching.

2. A catheter (1; 9; 13)
having a catheter tube (2) made of plastic with a closed catheter tip (7; 12),
having a passage opening (4) in a sheath wall section adjacent to the catheter tip (7; 12),
wherein the catheter tip (7; 12) is formed on an end section (8; 10; 14) of the catheter tube (2) adjacent to the catheter tip (7; 12),
wherein the catheter tip (7; 12) is made of a plastic material the Shore hardness of which is lower than that of the adjacent end section (8; 10; 14),
wherein in a region of the end section (8; 10; 14), a diameter of the catheter tube (2) is reduced compared to the other catheter tube (2) having an inner lumen (4a),
wherein the inner lumen (4a) of the catheter tube (2) is configured to be closed in the region of the end section (8).

3. The catheter according to claim 1, wherein the catheter tube (2) is made of a thermoplastic polyurethane elastomer (TPU) or of polyurethane (PUR) or of PVC.

4. The catheter according to claim 1, comprising a slidable coating of the catheter tube (29).

5. The catheter according to claim 1, wherein a Shore hardness of the catheter tip (7; 12) is in the range between 40 Shore A and 70 Shore A.

6. The catheter according to claim 1, wherein a Shore hardness of the adjacent end section (8; 10; 14) is in the range between 60 Shore A and 90 Shore A.

7. The catheter according to claim 1, wherein the end section (8; 10; 14) is made of drawn material of the catheter tube (2).

8. The catheter according to claim 1, wherein the end section (10; 14) is pressed to increase its circumference.

9. The catheter according to claim 1, wherein the end section (14) has a passage opening (15).

10. The catheter according to claim 1, wherein the catheter tip (12) is formed as an overmolding component around the end section (10; 14).

11. A method for manufacturing a catheter (1; 9; 13)
having a catheter tube (2) made of plastic with a closed catheter tip (7; 12),
having a passage opening (4) in a sheath wall section adjacent to the catheter tip (7; 12),
wherein the catheter tip (7; 12) is formed on an end section (8; 10; 14) of the catheter tube (2) adjacent to the catheter tip (7; 12),
wherein the catheter tip (7; 12) is made of a plastic material the Shore hardness of which is lower than that of the adjacent end section (8; 10; 14),
wherein in a region of the end section (8; 10; 14), a diameter of the catheter tube (2) is reduced compared to the other catheter tube (2) having an inner lumen (4a),
wherein the inner lumen (4a) of the catheter tube (2) is configured to be closed in the region of the end section (8),
comprising the following steps:
extruding the catheter tube (2),
closing the end section (8; 10; 14),
molding on the catheter tip (7; 12).

12. The method according to claim 11, wherein the molding on is performed by overmolding.

13. The method according to claim 11, wherein the closing of the end section (8; 10; 14) takes place when the extruded material of the catheter tube (2) is drawn out.

14. The method according to claim 11, comprising hot-pressing the end section (10; 14) before molding on the catheter tip (12).

15. The method according to claim 11, comprising a punching of the end section (14) before molding on the catheter tip (12).
